(19) United States
(12) Patent Application Publication
(10) Pub. No.: US 2010/0074407 A1
(43) Pub. Date: Mar. 25, 2010

(54) Treatment of lesions or imperfections in skin, near-skin or in other anatomic tissues, including under direct visualization

(76) Inventors: Steve Axelrod, Los Altos, CA (US); Thomas W. Rusch, Hopkins, MN (US); Paul A. Lovoi, Saratoga, CA (US); Anuradha Walawalkar, Sunnyvale, CA (US); Sara Z. Yao, San Mateo, CA (US)

Correspondence Address: Thomas M. Freiburger, P.O. Box 1026, Tiburon, CA, 94920, US

(21) Appl. No.: 12/284,289

(22) Filed: Sep. 19, 2008

(51) Int. Cl. A61N 5/10

(52) U.S. Cl. 378/65

(57) ABSTRACT

An apparatus and method for treatment of lesions or imperfections in or near exposed anatomic surfaces using low-level ionizing radiation includes a substantially transparent applicator to administer radiation from an energy source to a surface area with the lesion. The applicator is positioned over the lesion to be treated, a treatment plan is created to achieve the desired therapeutic effect to the lesion, and execution of the treatment plan is executed by the energy source. Verification of the treatment to plan and safety methods are disclosed.

### BACKGROUND OF THE INVENTION

This invention relates to the field of radiation therapy by means of ionizing radiation energy applied to tumors or other imperfections in skin or near-skin tissues, or in or near other exposed anatomical surfaces.

For many years, a variety of skin or near skin medical conditions have been treated by application of superficial voltage (50-150 kV) or orthovoltage (150-500 kV) x-ray therapy. These methods have drawn criticism because their penetration depth often puts deeper tissues at risk. As a result, external electron-beam radiation has come into greater use because its characteristic penetration can more easily be controlled. Such radiotherapy often follows surgery to excise a tumor or other defect. Often, the radiation apparatus used for electron beam treatment purposes is large, unwieldy, and incapable of being directed to the lesion with precision. Because it is designed to deliver high-energy radiation, the patient usually must be extensively shielded except for the area of the tumor, and radiation safety may require such apparatus only be operated from within a shielded room or “bunker”, from which all personnel other than the patient must be excluded during active treatment. This creates a situation which can be highly intimidating or claustrophobic for the patient. Because of the capital investment required for such an installation, this sort of treatment is often unavailable in small clinic or office situations. Reduction in capital expenditure requirements and intimidation factors would be great improvements over current external beam practice.

Furthermore, the available radiation output levels from such equipment may greatly exceed those required for the intended treatment, and output patterns are generally fixed. These output characteristics usually necessitate that the tumor be covered with radiation absorbing material such that the radiation intensity incident on the skin is more appropriate for the desired treatment. In addition, any necessary collimation of the radiation pattern to accommodate the patient's treatment requirements would be accomplished by conventional methods known to those of skill in the art.

In use, the machine is positioned properly over the patient to assure protection of adjacent normal tissues, with the collimated beam passing through secondary shielding which is directed at the subject tumor site. The patient must not move once alignment is determined, otherwise the treatment is not delivered to the desired area. When all is ready, the therapist leaves the room, and treatment is initiated. Because of the number of parameters in play to produce a satisfactory treatment and the complexity of the treatment setup, the potential for error and therefore imprecision in dosage delivery is significant. Clearly, smaller apparatus specifically designed for treatment of lesions and not requiring such extensive setup, treatment expense or safety precautions would be preferred to the apparatus and methods described above.

Contact applicators, usually including tungsten shielding, have been developed which utilize isotope (usually iridium) radiation sources (See Nucletron, Columbia, Md. 21046.) These applicators are difficult to position accurately over the lesion or imperfection to be treated, and as with high energy x-ray treatment, the characteristic penetration depth of iridium may well compromise underlying tissue. Use of isotopes like iridium in this manner will also require that treatment be carried out within a bunker as described above.

It is an object of this invention to provide methods for identifying the region to be treated on the patient, planning the radiation therapy quantitatively to be administered within that region, and executing the therapy conveniently, and in a timely manner. A further object of this invention is to provide a method of treatment of surface lesions and imperfections utilizing x-rays having controlled, minimal depth of penetration and which eliminate many of the safety concerns common to prior art methods as described above. A further object is to provide a convenient applicator which can be positioned over the lesion or imperfection to be treated under direct visualization, assuring proper treatment of diseased tissues, but sparing adjacent tissues. Still further, it is an object of this invention to provide a record of the therapy delivered, and to verify that the treatment was to plan.

### SUMMARY OF THE INVENTION

Specifically, this invention encompasses methods for treatment of skin or near-skin lesions or imperfections and is directed to the use of small ionizing radiation sources, preferably miniature x-ray sources comprising elements such as those described in U.S. Pat. Nos. 6,319,188, 7,127,033 and/or 7,158,612, all of which facilitate such radiation therapy in small clinical settings and are herein incorporated in their entirety by reference. The preferred x-ray energy level is in the range of up to about 50 kV, with preferred treatment depths between the tissue surface and 5 mm depth. Such x-ray sources can be modulated with regard to penetration depth by varying x-ray tube voltage, and to incident dose intensity by varying tube current, although in practice, it may be preferable to adjust exposure time rather than varying tube current to achieve a prescribed absorbed dose. These sources can in general be switched on and off as needed. The principles underlying such tubes may be found in Atoms, Radiation and Radiation Protection,

Second Edition, by James E. Turner, printed by John Wiley & Sons, 1995, incorporated herein in its entirety by reference.

The preferred embodiment of this invention further comprises an applicator incorporating provision for positioning the radiation beam over the region to be treated under direct visualization. The applicator comprises a base, preferably of stainless steel, and optionally a thin polymeric cover on the working side of the base, having a generally planar distal face substantially in contact with the skin. In the center of the base is a window of a size and shape to expose the lesion to the radiation emitted from within the applicator. The window size and shape may be chosen by the therapist to suit the therapeutic needs of the patient, via interchangeable bases. Except for the window, the base is substantially radio-opaque. We have found a thickness of stainless steel adjacent the window on the order of 2 mm adequate to provide sufficient attenuation in a typical therapeutic situation. More highly attenuating materials, for example tantalum, would permit thinner sections having the same levels of attenuation.

Proximal of the base of the preferred embodiment is an optically transparent housing preferably of leaded acrylic polymer (Nelco, Woburn, Mass.), or equivalent, through which the therapist may view the window overlying the lesion to achieve the alignment desired for therapy delivery. The attenuation of the leaded acrylic is preferably about 1/24 that of solid lead for equivalent thickness. The thickness of the acrylic housing therefore needs to be on the order of about 6 mm to sufficiently attenuate the back-scattered radiation. In positioning the applicator the therapist's view may be distorted, but it is still easy to observe and assure that the window circumscribes the desired treatment area. In an alternative embodiment without direct visualization, the housing may be of stainless steel or other materials as is further discussed below. In other respects, the embodiments are similar. In all embodiments described herein, it is preferable that elements in contact with or near the patient be capable of convenient sterilization, or be one-time-use components.

The base and housing generally share a common axis which is substantially transverse to the area of skin to be treated. Proximal of the housing is a cup-shaped filter of a low Z material, for example aluminum (or other materials in accordance with the isodose shaping discussion below), and proximal of that, a tubular source guide leading to other treatment system elements including a source power supply and controller. The source guide contains the source on the end of its cable and any utilities or support functions necessary to operate the x-ray tube, such as the coolant and cooling apparatus necessary to cool the x-ray source tube. See U.S. Pat. No. 7,127,033.

The aluminum filter may be contoured so as to shape the isodose patterns (imaginary surfaces of constant dose intensity), for example to flatten them, at the window where the radiation is incident on the skin, as well as distal of the applicator within the patient's body at or about the prescribed treatment depth. Further, the filter serves to strip off any low energy portions of the x-ray tube spectrum, hardening the beam in a conventional manner. The low energy elements of the unhardened spectrum are generally absorbed by the skin or other tissue which the beam initially encounters. With a hardened beam lacking these low energy components, the dose absorbed by the skin is reduced which may have cosmetic advantages or may even prevent substantial tissue inflammation.

In instances where the prescription is specified as a skin dose, alternate filter materials, for example silver or molybdenum, can also be designed to shape the x-ray intensity patterns as described above. We have found that filters of these materials essentially do not harden the beam and leave the energy spectrum substantially unaltered. That means a similar beam shaped by a silver or molybdenum filter is more quickly absorbed at the skin with less energy absorbed at depth. Such attenuators are discussed in more detail in co-pending U.S. patent application Ser. No. 12/072,620, and that application is incorporated herein in its entirety by reference.

When assembled and positioned properly over the lesion to be treated, the applicator may be held in position manually, clamped with respect to the operating table upon which the patient is lying, clamped with respect to the patient and his/her lesion, or otherwise supported.

In an alternate embodiment, the base and/or window element may be eliminated, and the leaded acrylic housing may be in direct contact with the skin surrounding the lesion.

In a further preferred embodiment, the applicator is similar to either of the descriptions above, but without optically transparent elements and therefore without provision for direct visualization. The applicator can optionally include targeting or reference marks which can be aligned with marks or features on the patient near the lesion to be treated.

In a still further embodiment, the applicator can be used in conjunction with a flexible shield (see U.S. patent application publication No. 2007/0075277, for example, incorporated herein in its entirety by reference) having a window of the desired shape which is positioned on the patient to expose the lesion to be treated, and to protect adjacent tissue. The applicator is then placed over the window and held in position as described above.

In all embodiments described above, the shape of the treatment window can be custom shaped to suit a given patient or lesion, or may be a member of a standard set of window shapes supplied as a kit. In the case of the flexible shield which can for example be of tungsten-loaded silicone rubber, a variety of silk-screened window patterns may optionally be printed on the sheet as supplied, such that the therapist can cut out a window in the shield to suit the case at hand.

Furthermore, a beam shaping filter can be used in conjunction with the applicator embodiments described above to harden or not harden the transmitted x-ray beam, and/or to shape the isodose patterns to suit the prescription for the case at hand. The applicator can also be instrumented to control the progression of the therapy, including in real time, or to create a record of the therapy delivered for inclusion in the patient's records. These and other objects, features and advantages of the invention will be apparent from the attached drawings and description of preferred embodiments which follow.

### DESCRIPTION OF THE DRAWINGS

FIG. 1 depicts an applicator of a preferred embodiment of the invention in longitudinal (elevational) cross section.

FIG. 2 depicts an applicator similar to that of FIG. 1 instrumented with a radiation sensor that can be in communication with a central controller, and with a radio-chromic film used to create a record of the treatment delivered.

FIG. 3 shows an alternate embodiment to that of FIG. 1.

FIGS. 4a, b, and c show schematically various filters used to shape the x-ray beam emitted when used in conjunction with embodiments of the invention.

FIGS. 5a and b show various silk-screened window patterns that can be placed on the surface of a flexible shield used in conjunction with applicators and/or methods of the invention.

### DESCRIPTION OF PREFERRED EMBODIMENTS

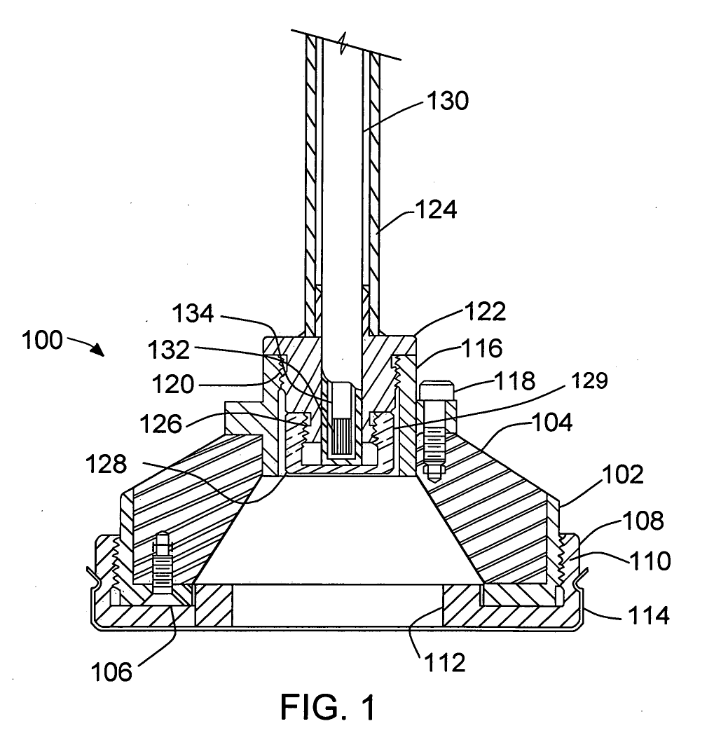

FIG. 1 shows a preferred applicator 100 of the invention comprising a base 102 which may be of stainless steel fastened on its proximal side to the distal end of a lead-loaded acrylic housing 104, such as by screws 106. Distal of the base is a preferably detachable window element 108, preferably also of stainless steel and secured to the circular base 102 by screw threads 110 (the window element/base combination can be referred to as a “base” or “base end”, although the two preferably are separable components). The window 112 limits the projection of the x-ray beam incident on the patient's skin to the shape of the window. Although the circular (cylindrical) base with screw threads is preferred, other shapes can be used, with other securing means.

Note also, the removable (and optionally interchangeable) window element 108 could be secured directly to the housing 104 if desired, by threads, machine screws, snap-on or otherwise.

An optional x-ray-transparent, thin polymeric cover 114 is snapped onto the distal face of the window element 108. This cover provides a level of cleanliness, and can be used to retain a radio-chromic film (not shown) as is discussed below. In use, the window element 108 (or cover 114, if present) is in contact with the patient's skin and positioned such that the lesion to be treated is exposed in the window 112. When in contact with the skin as described, the base and window element control both the shape and size of the incident beam onto the patient's skin, and substantially attenuate the beam elsewhere.

Proximal of the acrylic housing 104 is a flanged sleeve 116, the flange of which is used to secure the flanged sleeve to the acrylic housing 104 by screws 118. Internally at its proximal end, the flanged sleeve 116 is fastened by screw threads 120 to an adapter 122. The adapter serves several purposes. At its proximal end, the adapter 122 is permanently secured to the distal end of a source guide 124 as shown, by brazing, welding or silver soldering. Other conventional fastening methods, including by non-permanent screw-threads could also be used. The source guide could be non-metal.

The adapter 122 extends distally through the lumen of the flanged sleeve 116 to external threads 126 used to mount a cup-shaped radiation filter 128 which has an internally threaded ring or rim 129. Details of the radiation filter are discussed in greater detail below. If desired to protect the acrylic housing from undue radiation exposure, the distal end of the flanged sleeve 116 may be extended or configured relative to the filter in order to form an aperture limiting the spread of radiation within the housing such that radiation directly incident on the internal surface of the housing is reduced or eliminated.

Note that the assembly shown and described constitutes one preferred way of connecting the source guide 124 to the housing 104 and base 102; other types of assemblies can be employed, preferably providing for a filter 128, most preferably an interchangeable filter.

Through the source guide 124, a source catheter 130 is advanced until it abuts the inner side of the face of the radiation filter 128. Within the source catheter are the source 132 mounted on its cable 134, and any necessary x-ray tube utilities or apparatus necessary to support its proper functioning.

In use, the therapist selects the window (from a plurality of sizes and/or shapes) appropriate to the case at hand and determines the prescription to be delivered. A series of different window elements 108 can be provided. If treatment merely consists of an x-ray intensity level and treatment time, the necessary parameters are input to the system for delivery of the prescription. If the prescription is more complicated, the necessary settings for treatment delivery may be preset or noted in a convenient manner for manual control. Preferably, the treatment plan may be entered into an automated controller (not shown) as part of a treatment system such that once initiated, the prescribed treatment is delivered automatically.

Next, the elements of the applicator and source are assembled, and made ready for treatment delivery. The assembled apparatus is positioned over the skin area to be treated. By viewing the skin area through the housing (at least a portion of which is substantially optically transparent in a preferred embodiment), the operator correctly locates the applicator over the lesion to be treated. The applicator is then clamped in place, or alternatively, held in position manually, and treatment is initiated. At the conclusion of treatment, the x-ray emissions are switched off, and the apparatus is removed from the patient.

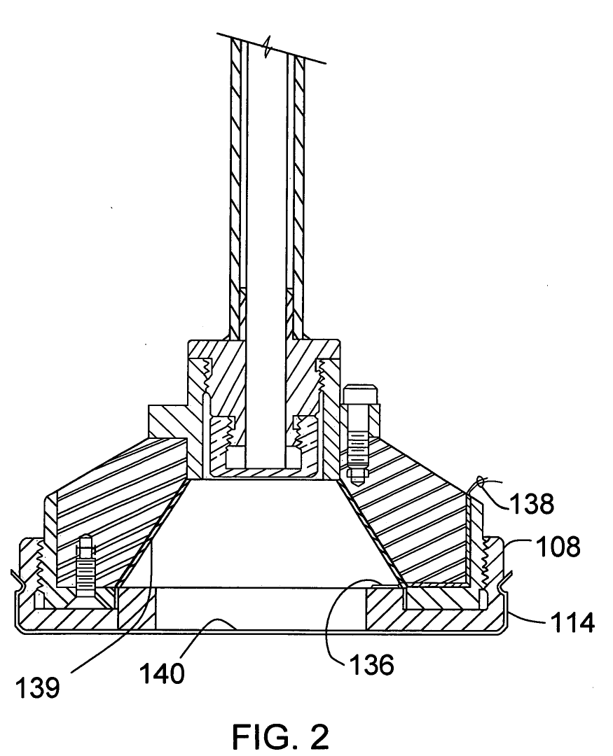

FIG. 2 shows the applicator of FIG. 1, further comprising a radiation sensor 136, which may be of the MOSFET type, mounted in the x-ray beam adjacent to the window of the window element 108. Conventional wiring 138 is routed through passages in the applicator apparatus for connecting the sensor to treatment system elements (not shown). Depending on the treatment system design, the sensor can be used to control through feedback x-ray tube output following the treatment plan, to create a record of treatment delivered, or to provide a safety cut-off if radiation should exceed the levels permitted by the prescription. Use of radiation sensors in this manner is disclosed in greater detail in U.S. Pat. No. 7,322,929, which is incorporated herein by reference in its entirety.

FIG. 2 further depicts a radio chromic film element 140 held in position between window element 108 and the polymeric cover 114. Such film (preferably GAF chromic type EBT film, International Specialty Products, Inc., Wayne, N.J.) is responsive to incident radiation, and after exposure can be used as a record of the treatment delivered for inclusion in the patient's medical record. In order to provide film orientation with respect to the lesion being treated, match marks or keying features relative to known apparatus orientation can be provided to uniquely record treatment delivered. Such orienting techniques are familiar to those skilled in the art.

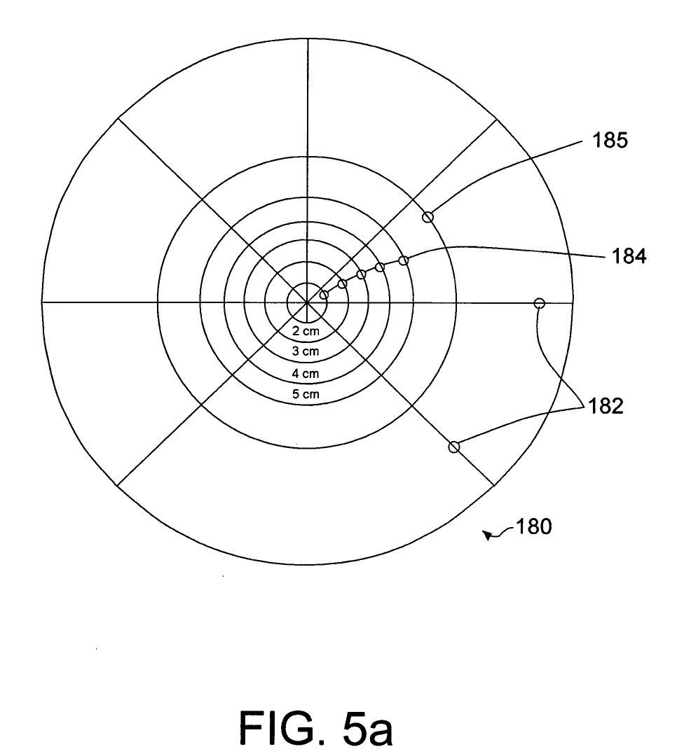

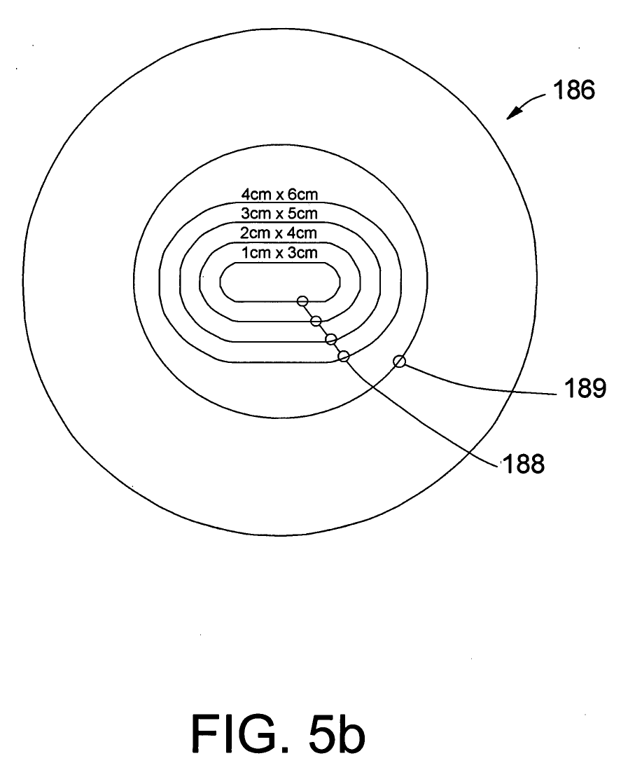

FIG. 2 also shows a liner 139 inside an alternate, opaque housing. These elements are discussed in detail in connection with the description of FIG. 5a below.

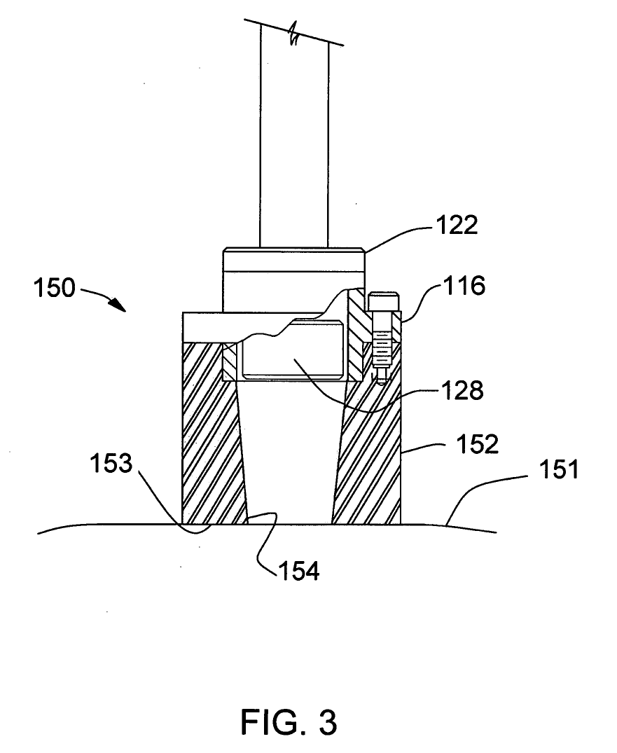

FIG. 3 shows an alternate applicator apparatus embodiment 150 in which the housing 152 is in direct contact with the patient's skin 151. In other respects, the flanged sleeve 116, adapter 122, and filter 128 are substantially the same as described with reference to FIG. 1. In this case the base end of the applicator is the skin-contacting end 153 of the housing 152. As shown, the housing limits the x-ray beam incident on the skin to a small area rather than larger as shown in FIG. 1. In principle, however, window size and housing shape are independent of other features provided in either of the embodiments. Preferably, the housing 152 is transparent to visible light as in the embodiment of FIG. 1, and again, the window 154 is positioned over the skin lesion under direct visualization (with angled viewing through the housing 152).

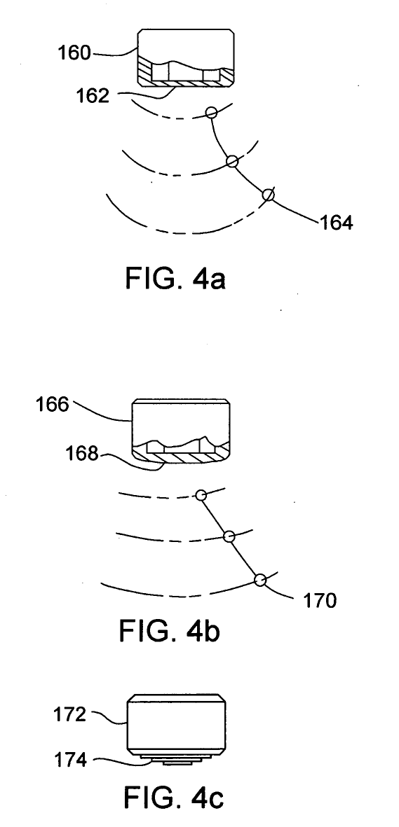

FIGS. 4a, b and c show variations in filter designs, and in general, the differences in isodose shapes which result. FIG. 4a depicts a filter 160 having a flat, uniformly thick face 162 which attenuates uniformly. That is, the beam intensity is reduced, but its isodose surfaces 164 are substantially the same as those emanating from the unfiltered source. FIG. 4b shows a similar filter 166 having a face 168 which is thicker in the middle than at its edges, and having the effect of flattening the shape of the isodose surfaces 170. Flattening the isodose surfaces at the skin or treatment depth is a common objective in radiotherapy. It is clear that the degree of flattening is correlated with the filter face 168 contour, and that simple experimentation can be used to flatten or otherwise shape the isodose surfaces of sources having various emission characteristics. We have also found that stepped variations in filter face surfaces which approximate similar but continuously shaped faces produce substantially the same isodose surfaces. Such a stepped face is shown in FIG. 4c where the filter 172 has a stepped face 174. The isodose surfaces produced are very similar to those surfaces 170 of FIG. 4b.

As described in the summary above, and in the aforementioned co-pending application Ser. No. 12/072,620, most low Z filter materials, such as aluminum for example, serve to conventionally harden the x-ray beam, that is they strip off low-energy portions of the emitted spectrum, which tightens the energy spectrum of what is transmitted through the filter. Since energy equates to penetration depth, such filters can be tuned to target a narrow penetration depth, thereby reducing the dose absorbed at the skin. As described in the referenced application, we have found that a few filter materials, notably silver and molybdenum, have little effect on the energy distribution of the transmitted beam, and can be effectively used where a skin dose is required, and to reduce the dose absorbed at depth. The beam shaping principles are the same with either sort of materials, but the ranges over which they are most useful to the therapist differ.

FIG. 5a shows in plan view, a flexible shield 180 of tungsten-loaded silicone rubber, or as might otherwise be described in co-pending U.S. patent application No. 2007/0075277. Silk-screened on the surface is an exemplary pattern of radial lines 182 emanating from a center position which can be used for orienting the shield, and concentric, circular pattern of lines 184 which can be used by the therapist as a guide for cutting windows responsive to the patient's prescription and needs. When so prepared, the shield can be used to form a treatment window rather than using the window element 108 described above. Such use may eliminate the need for direct visualization through the applicator housing 104 or housing 152 during placement since the shield and window can be placed and secured over the lesion first, followed by positioning of the applicator using the silk screened lines on the shield for guidance. The outer silk-screened circle 185 may be sized to correspond with the outside diameter of the window element 108 of the applicator 100, or of the polymer cover 114 if present (see FIG. 1), and used to position the applicator.

Without the need for visualization, the preferred applicator embodiment would be as shown in FIG. 1, except the housing may now be metallic, for example, stainless steel as described earlier. Further, it may be advantageous if the housing inner surface is of a low Z material like aluminum, since the portion of the x-ray beam incident on the housing inner surface will be at a small angle, Compton scatter may be expected and such scattered radiation will generally be directed at the edges of the window. Such scattered energy will therefore have an effect similar to a flattening filter, perhaps even eliminating the need for such a filter. A housing inner liner 139 is shown in FIG. 2 and can be a machined part, for example, positioned within the housing at assembly, or alternatively, such a low Z layer can be added onto the housing by a conventional vapor deposition process.

A flexible shield 180 (FIG. 5a) can also be used in conjunction with the applicator embodiments of FIGS. 1 and 3, and since the shield contributes importantly to the overall attenuation, including shielding the tissue outside the applicator itself, the thickness (hence attenuation value) of the applicator elements can likely be reduced making the shield/applicator combination more ergonomically attractive.

FIG. 5b depicts a flexible shield 186 similar to that of FIG. 5a, but having an exemplary silk-screened pattern of concentric oval (racetrack shaped) lines 188 to facilitate cutting oval masks. Other patterns may also be used if the level of need justifies the differentiation. Again, as with the circle 185 of FIG. 5a, the outer silk-screened circle 189 may be sized to assist in positioning the applicator with respect to the oval window formed in the shield 186.

Several different combinations of features have been incorporated in the different embodiments described above. In principle, these may be included in different combinations, or the elements arranged in different configurations relative to one another but such that the functions of each are still effective.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

We claim:

1. A contact applicator for administering radiation to a patient's skin or near-skin tissues, comprising:
a base end with a distal, skin-contacting surface,
a housing extending up proximally from the base end,
a window within the base end through which radiation can be delivered to the patient's tissue, and
a source guide connected to the housing and positioned proximal of the base end and generally aligned with the window, with an electronic, controllable x-ray source positioned in the source guide to direct radiation through the window and to the patient's tissue.

2. The applicator of claim 1, wherein at least a portion of the housing is substantially optically transparent, allowing direct visualization of the patient's skin through the housing for proper positioning and alignment of the applicator.

3. The applicator of claim 1, wherein the distal, skin-contacting surface of the base is substantially planar.

4. The applicator of claim 1, further including a removable snap-on cover configured to be attached over the distal end of the base.

5. The applicator of claim 4, wherein the cover for the base is formed of substantially x-ray-transparent polymer, such that the cover is put in contact with a patient's skin during treatment.

6. The applicator of claim 1, wherein the housing and the base end are a single, integral component.

7. The applicator of claim 1, wherein the base end is secured to the housing and of a different material than the housing, which is substantially optically transparent, thus allowing direct visualization of the patient's skin through the housing for proper positioning and alignment of the applicator.

8. The applicator of claim 1, wherein the base end includes a window element providing the window through which radiation is directed.

9. The applicator of claim 8, including a series of interchangeable said window elements separately attachable to the remainder of the base end, the window elements defining different sizes or shapes of windows.

10. The applicator of claim 9, wherein each of the series of window elements has a generally cylindrical, internally-threaded opening for securing on external threads on the remainder of the base end.

11. The applicator of claim 1, wherein at least a portion of the base end is substantially radio-opaque to minimize radiation exposure on patient tissue areas surrounding the window.

12. The applicator of claim 1, wherein the housing includes, proximal of the window, a hollow space such that the source guide and radiation source are spaced substantially away from the patient's skin.

13. The applicator of claim 2, wherein the substantially optically transparent housing is comprised of leaded acrylic polymer, so as to attenuate backscattered radiation.

14. The applicator of claim 1, further including, connecting the source guide to the housing, a sleeve secured to the housing and an adapter connected within the sleeve, the adapter supporting the source guide generally centrally within the adapter.

15. The applicator of claim 14, further including a radiation filter secured to the adapter so as to be positioned just distally of the source guide for filtering radiation from the x-ray source.

16. The applicator of claim 15, wherein the filter is cup-shaped, with an upwardly extending internally threaded rim connected to external threads of the adapter.

17. The applicator of claim 16, wherein the filter is removable from the adapter, and with a plurality of different filters selectable for use on the adapter.

18. The applicator of claim 1, further including a radiation filter connected to the housing and positioned to filter radiation directed toward the window and the patient's skin.

19. The applicator of claim 18, wherein the radiation filter is cup-shaped and removable from the base, with a plurality of different filters selectable for use on the adapter.

20. The applicator of claim 1, further including a radiation sensor secured to the housing or base end in the path of radiation from the electronic x-ray source, connectable to treatment system elements for monitoring radiation administered to the patient during treatment with the applicator.

21. A method for treating a patient's skin or near-skin tissues with radiation, comprising:
selecting a contact applicator having a base end with a skin-contacting surface, a housing extending up proximally from the base end, a window in the base end through which radiation can be delivered to the patient's tissue and a source guide connected to the housing proximal of the base end,
positioning the contact applicator as desired over a lesion or imperfection in the skin or near-skin tissues, by viewing the skin through the housing of the contact applicator and through the window, at least a portion of the housing being substantially optically transparent, and
with an electronic, controllable x-ray source positioned in the source guide, switching on the x-ray source and directing radiation through the window to the patient's skin or near-skin tissues to treat lesions or imperfections in the skin or near-skin tissues.

22. The method of claim 21, wherein the applicator additionally includes a radiation sensor in the path of radiation from the x-ray source, and the method including monitoring radiation delivered to the skin during treatment via the radiation sensor.

23. The method of claim 22, further including controlling the radiation delivered from the electronic, controllable x-ray source in real time during treatment of the patient, using feedback from the radiation sensor and in response to such feedback, controlling the output of the electronic x-ray source.

24. The method of claim 21, further including, prior to the step of positioning the contact applicator, selecting a said window for the base end from a plurality of windows of different sizes or shapes, as appropriate for the particular lesion or imperfection to be treated, and securing the selected window to the applicator.

25. The method of claim 21, further including, prior to the step of positioning the contact applicator, selecting a radiation filter appropriate for the particular treatment and securing the filter into the applicator generally adjacent to and distal of the source guide so as to be in the path of radiation.

26. The method of claim 21, further including placing a radio chromic film element in the window prior to positioning the contact applicator, and, after completion of the radiation treatment, using the radiation-exposed radio chromic film element as a record of the treatment delivered, and including the film element in the patient's medical record.
